(19) United States
(12) Patent Application Publication
(10) Pub. No.: US 2020/0377914 A1
(43) Pub. Date: Dec. 3, 2020

(54) EXTRACTION OF ALKANOIC ACIDS

(71) Applicant: Evonik Operations GmbH, Essen (DE)

(72) Inventors: Thomas HAAS, Muenster (DE); Simon BECK, Muenster (DE); Martin DEMLER, Dorsten (DE)

(73) Assignee: Evonik Operations GmbH, Essen (DE)

(21) Appl. No.: 16/969,853

(22) PCT Filed: Feb. 15, 2019
(86) PCT No.: PCT/EP2019/053786

§ 371 Date: Aug. 13, 2020

(30) Foreign Application Priority Data
Feb. 15, 2018 (EP) 18156841.1

(51) Int. Cl. C12P 7/40; C12P 7/64; B01D 11/04

(52) U.S. Cl. CPC C12P 7/40; B01D 11/0492; C12P 7/6409

(57) ABSTRACT

The present invention relates to a method of extracting an alkanoic acid and/or ester there of from an aqueous medium, the method comprising: (a) contacting the alkanoic acid and/or ester thereof in the aqueous medium with at least one extracting medium for a time sufficient to extract the alkanoic acid and/or ester thereof from the aqueous medium into the extracting medium, (b) separating the extracting medium with the extracted alkanoic acid and/or ester thereof from the aqueous medium wherein the extracting medium comprises: -a mixture of at least one alkyl-phosphine oxide, preferably Trioctylphosphine oxide (TOPO), and at least one alkane wherein the alkane comprises at least 12 carbon atoms.

### FIELD OF THE INVENTION

The present invention relates to a method for extracting an alkanoic acid and/or ester thereof from an aqueous medium. In particular, the method uses a mixture of at least one alkyl-phosphine oxide, preferably Trioctylphosphine oxide (TOPO), and at least one alkane.

### BACKGROUND OF THE INVENTION

Alkanoic acids are carboxylic acids in which an oxygen atom (═O) has been substituted for two of the hydrogen atoms in the corresponding alkane, and, an OH functional group has substituted for another H atom on the same carbon atom. Alkanoic acids have several functions in the art. For example, they can be used in the production of polymers, pharmaceuticals, solvents, and food additives.

A well-known process for preparing and extracting alkanoic acids involves the hydrolysis and decarboxylation of malonic esters. The malonic ester is saponified using aqueous sodium hydroxide to result in the formation of an aqueous solution of disodium salt and ethanol. The salt solution is then treated with a strong mineral acid to produce a mineral acid sodium salt and to precipitate the solid dicarboxylic acid. Simple separation procedures such as filtration or extraction, is used to then isolate the dicarboxylic acid. The sodium salt is discarded as waste. The isolated acid is further dried and heated to a temperature sufficient to cause decarboxylation to occur. This procedure is lengthy, requires numerous steps, generates waste, and is equipment intensive.

Another method for extracting alkanoic acids such as formic, acetic, propionic, lactic, succinic, and citric acids is a salting-out extraction. This method uses a system composed of ethanol and ammonium sulfate. The system parameters influencing the extraction efficiency, include tie line length, phase volume ratio, acid concentration, temperature, system pH and the like. Although the extraction efficiency of alkanoic acids was shown to increase using this method, the various parameters involved makes the method too complicated for industrial use.

CA1167051 discloses a method of extracting or recovering some carboxylic acids such as acetic acid and formic acid. However, the method requires the use of high temperatures and special equipment for the steps of counterflow heat exchanging.

Accordingly, there is a need in the art for a cheaper and more efficient extraction method for extracting alkanoic acids, especially alkanoic acids produced in industrial scale. Further, there is a need for an extraction method of alkanoic acids that can be used in connection with a biotechnological method of producing the alkanoic acids.

### DESCRIPTION OF THE INVENTION

The present invention attempts to solve the problems above by providing a means of extracting alkanoic acids and/or ester thereof that is more efficient and cheaper than the current methods available in the art. The present invention also provides a means of extracting alkanoic acids and/or ester thereof that can be used in conjunction with a biotechnological method of producing alkanoic acids and/or ester thereof.

According to one aspect of the present invention, there is provided a method of extracting an alkanoic acid and/or ester thereof from an aqueous medium, the method comprising:
(a) contacting the alkanoic acid and/or ester thereof in the aqueous medium with at least one extracting medium for a time sufficient to extract the alkanoic acid and/or ester thereof from the aqueous medium into the extracting medium,
(b) separating the extracting medium with the extracted alkanoic acid and/or ester thereof from the aqueous medium
wherein the extracting medium comprises:
a mixture of at least one alkyl-phosphine oxide, preferably Trioctylphosphine oxide (TOPO), and at least one alkane
wherein the alkane comprises at least 12 carbon atoms.

In particular, the extraction method according to any aspect of the present invention allows for an increase in yield relative to the amount of extractants used. For example, less than 50% by weight of extracting medium may be used to extract the same amount of alkanoic acids and/or ester thereof as if only pure alkanes were used. Therefore, with a small volume of extracting medium, a larger yield of alkanoic acids and/or ester thereof may be extracted. The extracting medium is also not harmful to microorganisms. Accordingly, the extracting medium according to any aspect of the present invention may be present when the alkanoic acid and/or ester thereof is biotechnologically produced. Further, at least when the alkanoic acid is a hexanoic acid, this can be easily separated from the extracting medium according to any aspect of the present invention by distillation. This is because hexanoic acid at least distills at a significantly lower boiling point than the extracting medium and after the separation via distillation, the extracting medium may be easily recycled.

The method according to any aspect of the present invention may be a method of extracting at least one isolated alkanoic acid and/or ester thereof from an aqueous medium. An isolated alkanoic acid and/or ester thereof may refer to at least one alkanoic acid and/or ester thereof that may be separated from the medium where the alkanoic acid and/or ester thereof has been produced. In one example, the alkanoic acid and/or ester thereof may be produced in an aqueous medium (e.g. fermentation medium where the alkanoic acid and/or ester thereof is produced by specific cells from a carbon source). The isolated alkanoic acid and/or ester thereof may refer to the alkanoic acid and/or ester thereof extracted from the aqueous medium. In particular, the extracting step allows for the separation of excess water from the aqueous medium thus resulting in a formation of a mixture containing the extracted alkanoic acid and/or ester thereof.

The extracting medium may also be referred to as the ‘extraction medium’. The extraction medium may be used for extracting/ isolating the alkanoic acid and/or ester thereof produced according to any method of the present invention from the aqueous medium wherein the alkanoic acid and/or ester thereof was originally produced. At the end of the extracted step, excess water from the aqueous medium may be removed thus resulting in the extracting medium containing the extracted alkanoic acid and/or ester thereof. The extracting medium may comprise a combination of compounds that may result in an efficient means of extracting the alkanoic acid and/or ester thereof from the aqueous medium. In particular, the extracting medium may comprise: (i) at least alkane comprising at least 12 carbon atoms, and (ii) at least one molecule alkyl-phosphine oxide. The extraction medium according to any aspect of the present invention may efficiently extract the alkanoic acid and/or ester thereof into the alkane- alkyl-phosphine oxide extracting medium. This extracting medium of a mixture of alkyl-phosphine oxide and at least one alkane may be considered suitable in the method according to any aspect of the present invention as the mixture works efficiently in extracting the desired alkanoic acid and/or ester thereof in the presence of a fermentation medium. In particular, the mixture of alkyl-phosphine oxide and at least one alkane may be considered to work better than any method currently known in the art for extraction of alkanoic acid and/or ester thereof as it does not require any special equipment to be carried out and it is relatively easy to perform with a high product yield.

The alkane may comprise at least 12 carbon atoms. In particular, the alkane may comprise at 12-18 carbon atoms. In one example, the alkane may be selected from the group consisting of dodecane, tridecane, tetradecane, pentadecane, hexadecane, heptadecane and octadecane. In a further example, the extracting medium may comprise a mixture of alkanes.

Alkyl-phosphine oxides have a general formula of OPX₃, where X is an alkyl. Suitable alkyl phosphine oxides according to any aspect of the present invention include an alkyl group composed of a linear, branched or cyclic hydrocarbon, the hydrocarbon composed of from 1 to about 100 carbon atoms and from 1 to about 200 hydrogen atoms. In particular, “alkyl” as used in reference to alkyl phosphine oxide according to any aspect of the present invention can refer to a hydrocarbon group having 1 to 20 carbon atoms, frequently between 4 and 15 carbon atoms, or between 6 and 12 carbon atoms, and which can be composed of straight chains, cyclics, branched chains, or mixtures of these. The alkyl phosphine oxide may have from one to three alkyl groups on each phosphorus atom. In one example, the alkyl phosphine oxide has three alkyl groups on P. In some examples, the alkyl group may comprise an oxygen atom in place of one carbon of a C4-C15 or a C6-C12 alkyl group, provided the oxygen atom is not attached to P of the alkyl phosphine oxide. Typically, the alkyl phosphine oxide is selected from the group consisting of tri-octylphosphine oxide, tri-butylphosphine oxide, hexyl-phosphine oxide, octylphosphine oxide and mixtures thereof.

Even more in particular, the alkyl phosphine oxide may be tri-octylphosphine oxide (TOPO). Trioctylphosphine oxide (TOPO) is an organophosphorus compound with the formula OP(C8H17)3. The at least one alkyl-phosphine oxide, preferably Trioctylphosphine oxide (TOPO), may be present in the extraction medium together with at least one alkane. In particular, the mixture of at least one alkyl-phosphine oxide, preferably Trioctylphosphine oxide (TOPO), and alkane comprising at least 12 carbon atoms may comprise about 1:100 to 1:10 weight ratio of at least one alkyl-phosphine oxide, preferably Trioctylphosphine oxide (TOPO), relative to the alkane. More in particular, the weight ratio of at least one alkyl-phosphine oxide, preferably Trioctylphosphine oxide (TOPO), to alkane in the extraction medium according to any aspect of the present invention may be about 1:100, 1:90, 1:80, 1:70, 1:60, 1:50, 1:40, 1:30, 1:25, 1:20, 1:15, or 1:10. Even more in particular, the weight ratio of at least one alkyl-phosphine oxide, preferably Trioctylphosphine oxide (TOPO), to alkane may be selected within the range of 1:90 to 1:10, 1:80 to 1:10, 1:70 to 1:10, 1:60 to 1:10, 1:50 to 1:10, 1:40 to 1:10, 1:30 to 1:10 or 1:20 to 1:10. The weight ratio of at least one alkyl-phosphine oxide, preferably Trioctylphosphine oxide (TOPO), to alkane may be between 1:40 to 1:15 or 1:25 to 1:15. In one example, the weight ratio of at least one alkyl-phosphine oxide, preferably Trioctylphosphine oxide (TOPO), to alkane may be about 1:15. In the example, the alkane may be hexadecane and therefore the weight ratio of at least one alkyl-phosphine oxide, preferably Trioctylphosphine oxide (TOPO), to hexadecane may be about 1:15.

The term ‘about’ as used herein refers to a variation within 20 percent. In particular, the term “about” as used herein refers to +/−20%, more in particular, +/−10%, even more in particular, +/−5% of a given measurement or value.

In step (a) according to any aspect of the present invention, the alkanoic acid and/or ester thereof in the aqueous medium may contact the extracting medium for a time sufficient to extract the alkanoic acid and/or ester thereof from the aqueous medium into the extracting medium. A skilled person may be capable of determining the amount of time needed to reach distribution equilibrium and the right bubble agglomeration that may be needed to optimize the extraction process. In some examples the time needed may be dependent on the amount of alkanoic acid and/or ester thereof that may be extracted. In particular, the time needed to extract the alkanoic acid and/or ester thereof from the aqueous medium into the extracting medium may only take a few minutes. In examples where the extraction is carried out as fermentation takes place, the time for extraction is equivalent to the time of fermentation.

The ratio of the extracting medium used to the amount of alkanoic acid and/or ester thereof to be extracted may vary depending on how quick the extraction is to be carried out. In one example, the amount of extracting medium is equal to the amount of aqueous medium comprising the alkanoic acid and/or ester thereof. After the step of contacting the extracting medium with the aqueous medium, the two phases (aqueous and organic) are separated using any means known in the art. In one example, the two phases may be separated using a separation funnel. The two phases may also be separated using mixer-settlers, pulsed columns, and the like. In one example, where the alkanoic acid is hexanoic acid, the separation of the extracting medium from the hexanoic acid may be carried out using distillation in view of the fact that hexanoic acid distills at a significantly lower boiling point than the extracting medium. A skilled person may be able to select the best method of separating the extraction medium from the desired alkanoic acid and/or ester thereof in step (b) depending on the characteristics of the alkanoic acid and/or ester thereof desired to be extracted.

In particular, step (b) according to any aspect of the present invention involves the recovering of the alkanoic acid from step (a). The alkanoic acid brought into contact with the organic extracting medium results in the formation of two phases, the two phases (aqueous and organic) are separated using any means known in the art. In one example, the two phases may be separated using a separation funnel. The two phases may also be separated using mixer-settlers, pulsed columns, thermal separation and the like. In one example, where the alkanoic acid is hexanoic acid, the separation of the extracting medium from the hexanoic acid may be carried out using distillation in view of the fact that hexanoic acid distills at a significantly lower boiling point than the extracting medium. A skilled person may be able to select the best method of separating the extracting medium from the desired alkanoic acid depending on the characteristics of the alkanoic acid desired to be recovered.

Step (b) preferably ends with the organic absorbent made available again to be recycled or reused, preferably in step (0) (see below).

The alkanoic acid and/or ester thereof may be selected from the group consisting of alkanoic acids with 2 to 16 carbon atoms. In particular, the alkanoic acid may be selected from the group consisting of ethanoic acid, propionic acid, butanoic acid, pentanoic acid, hexanoic acid, heptanoic acid, octanoic acid, nonanoic acid, decanoic acid, undecanoic acid, dodecanoic acid, tridecanoic acid, mystric acid, pentadecanoic acid and hexadecanoic acid. More in particular, the alkanoic acid may be selected from the group consisting of alkanoic acids with 4 to 16, 4 to 14, 4 to 12, 4 to 10, 5 to 16, 5 to 14, 5 to 12, 5 to 10, 6 to 16, 6 to 14, 6 to 12, or 6 to 10 carbon atoms. Even more in particular, the alkanoic acid is a hexanoic acid.

The ester part of the ester of the alkanoic acid is preferably chosen from the group consisting of methyl, ethyl, isopropyl, propyl and isobutyl and butyl.

In some examples, microorganisms capable of producing the alkanoic acid and/or ester thereof may be cultivated with any culture media, substrates, conditions, and processes generally known in the art for culturing bacteria. This allows for the alkanoic acid and/or ester thereof to be produced using a biotechnological method. Depending on the microorganism that is used for alkanoic acid and/or ester thereof production, appropriate growth medium, pH, temperature, agitation rate, inoculum level, and/or aerobic, microaerobic, or anaerobic conditions are varied. A skilled person would understand the other conditions necessary to carry out the method according to any aspect of the present invention. In particular, the conditions in the container (e.g. fermenter) may be varied depending on the microorganisms used. The varying of the conditions to be suitable for the optimal functioning of the microorganisms is within the knowledge of a skilled person.

In one example, the method according to any aspect of the present invention may be carried out in an aqueous medium with a pH between 5 and 8, or 5.5 and 7. The pressure may be between 1 and 10 bar. The microorganisms may be cultured at a temperature ranging from about 20° C. to about 80° C. In one example, the microorganism may be cultured at 37° C.

In some examples, for the growth of the microorganism and for its production of alkanoic acid and/or ester thereof, the aqueous medium may comprise any nutrients, ingredients, and/or supplements suitable for growing the microorganism or for promoting the production of the alkanoic acid and/or ester thereof. In particular, the aqueous medium may comprise at least one of the following: carbon sources, nitrogen sources, such as an ammonium salt, yeast extract, or peptone; minerals; salts; cofactors; buffering agents; vitamins; and any other components and/or extracts that may promote the growth of the bacteria. The culture medium to be used must be suitable for the requirements of the particular strains. Descriptions of culture media for various microorganisms are given in “Manual of Methods for General Bacteriology”.

Accordingly, the method of extraction of an alkanoic acid and/or ester thereof according to any aspect of the present invention may be used together with any biotechnological method of producing the alkanoic acid and/or ester thereof. This is especially advantageous as usually during the fermentation process to produce alkanoic acid and/or ester thereof using biological methods, the alkanoic acid and/or ester thereof would be left to collect in the aqueous medium and after reaching certain concentrations in the fermentation medium, the very target product (alkanoic acids and/or ester thereof) may inhibit the activity and productivity of the microorganism. This thus limits the overall yield of the fermentation process. With the use of this extraction method, the alkanoic acids and/or ester thereof are extracted as they are produced thus reducing end-product inhibition drastically.

The method according to any aspect of the present invention is also more efficient and cost-effective than the traditional methods of removing alkanoic acids and/or ester thereof, particularly from a fermentation method as they are produced, as there is no primary reliance on distillation and/or a precipitation for recovering of alkanoic acids and/or ester thereof. Distillation or precipitation process may lead to higher manufacturing costs, lower yield, and higher waste products therefore reducing the overall efficiency of the process. The method according to any aspect of the present invention attempts to overcome these shortcomings.

In one example, the alkanoic acid is hexanoic acid. In this example, the hexanoic acid may be produced from synthesis gas.

The synthesis gas may be converted to hexanoic acid in the presence of at least one acetogenic bacteria and/or hydrogen oxidising bacteria. In particular, any method known in the art may be used. Hexanoic acid may be produced from synthesis gas by at least one prokaryote. In particular, the prokaryote may be selected from the group consisting of the genus Escherichia such as Escherichia coli; from the genus Clostridia such as Clostridium ljungdahlii, Clostridium autoethanogenum, Clostridium carboxidivorans or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia such as Delftia acidovorans; from the genus Bacillus such as Bacillus subtillis; from the genus Lactobacillus such as Lactobacillus delbrueckii; or from the genus Lactococcus such as Lactococcus lactis.

In another example, hexanoic acid may be produced from synthesis gas by at least one eukaryote. The eukaryote used in the method of the present invention may be selected from the genus Aspergillus such as Aspergillus niger; from the genus Saccharomyces such as Saccharomyces cerevisiae; from the genus Pichia such as Pichia pastoris; from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis.

More in particular, hexanoic acid may be produced from synthesis gas by any method disclosed in Steinbusch, 2011, Zhang, 2013, Van Eerten-Jansen, M. C. A. A, 2013, Ding H. et al, 2010, Barker H. A., 1949, Stadtman E. R., 1950, Bornstein B. T., et al., 1948 and the like. Even more in particular, the hexanoic acid may be produced from synthesis gas in the presence of at least Clostridium kluyveri.

The term “acetogenic bacteria” as used herein refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway. In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii O-52 (ATCC 55989), Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui).

More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ER12, Clostridium ljungdahlii COL and Clostridium ljungdahlii O-52 may be used in the conversion of synthesis gas to hexanoic acid. These strains for example are described in WO 98/00558, WO 00/68407, ATCC 49587, ATCC 55988 and ATCC 55989.

The acetogenic bacteria may be used in conjunction with a hydrogen oxidising bacteria. In one example, both an acetogenic bacteria and a hydrogen oxidising bacteria may be used to produce hexanoic acid from synthesis gas. In another example, only acetogenic bacteria may be used for metabolising synthesis gas to produce hexanoic acid from synthesis gas. In yet another example, only a hydrogen oxidising bacteria may be used in this reaction.

The hydrogen oxidising bacteria may be selected from the group consisting of Achromobacter, Acidithiobacillus, Acidovorax, Alcaligenes, Anabena, Aquifex, Arthrobacter, Azospirillum, Bacillus, Bradyrhizobium, Cupriavidus, Derxia, Helicobacter, Herbaspirillum, Hydrogenobacter, Hydrogenobaculum, Hydrogenophaga, Hydrogenophilus, Hydrogenothermus, Hydrogenovibrio, Ideonella sp. O1, Kyrpidia, Metallosphaera, Methanobrevibacter, Myobacterium, Nocardia, Oligotropha, Paracoccus, Pelomonas, Polaromonas, Pseudomonas, Pseudonocardia, Rhizobium, Rhodococcus, Rhodopseudomonas, Rhodospirillum, Streptomyces, Thiocapsa, Treponema, Variovorax, Xanthobacter and Wautersia.

In the production of hexanoic acid from synthesis gas a combination of bacteria may be used. There may be more than one acetogenic bacteria present in combination with one or more hydrogen oxidising bacteria. In another example, there may be more than one type of acetogenic bacteria present only. In yet another example, there may more than one hydrogen oxidising bacteria present only. Hexanoic acid also known as caproic acid has general formula C₅H₁₁COOH.

In particular, the hexanoic producing method may comprise the step of:
contacting the synthesis gas with at least one bacteria capable of carrying out the Wood-Ljungdahl pathway and the ethanol-carboxylate fermentation to produce hexanoic acid.

The term “contacting”, as used herein, means bringing about direct contact between the alkanoic acid and/or ester thereof in the medium with the extraction medium in step (a) and/or the direct contact between the microorganism and synthesis gas. For example, the cell, and the medium comprising the carbon source may be in different compartments. In particular, the carbon source may be in a gaseous state and added to the medium comprising the cells according to any aspect of the present invention.

In one example, the production of hexanoic acid from synthesis gas may involve the use of the acetogenic bacteria in conjunction with a bacterium capable of producing the hexanoic acid using ethanol-carboxylate fermentation hydrogen oxidising bacteria. In one example, both an acetogenic bacteria and a hydrogen oxidising bacteria may be used to produce hexanoic acid from synthesis gas. For example, Clostridium ljungdahlii may be used simultaneously with Clostridium kluyveri. In another example, only acetogenic bacteria may be used for metabolising synthesis gas to produce hexanoic acid from synthesis gas. In this example, the acetogenic bacteria may be capable of carrying out both the ethanol-carboxylate fermentation pathway and the Wood-Ljungdahl pathway. In one example, the acetogenic bacteria may be C. carboxidivorans which may be capable of carrying out both the Wood-Ljungdahl pathway and the ethanol-carboxylate fermentation pathway.

The ethanol-carboxylate fermentation pathway is described in detail at least in Seedorf, H., et al., 2008. The organism may be selected from the group consisting of Clostridium kluyveri, C. Carboxidivorans and the like. These microorganisms include microorganisms which in their wild-type form do not have an ethanol-carboxylate fermentation pathway, but have acquired this trait as a result of genetic modification. In particular, the microorganism may be Clostridium kluyveri.

In one example, the bacteria used according to any aspect of the present invention is selected from the group consisting of Clostridium kluyveri and C. Carboxidivorans.

In particular, the cells are brought into contact with a carbon source which includes monosaccharides (such as glucose, galactose, fructose, xylose, arabinose, or xylulose), disaccharides (such as lactose or sucrose), oligosaccharides, and polysaccharides (such as starch or cellulose), one-carbon substrates and/or mixtures thereof. More in particular, the cells are brought into contact with a carbon source comprising CO and/or CO₂ to produce an alkanoic acid and/or ester thereof.

With respect to the source of substrates comprising carbon dioxide and/or carbon monoxide, a skilled person would understand that many possible sources for the provision of CO and/or CO2 as a carbon source exist. It can be seen that in practice, as the carbon source of the present invention any gas or any gas mixture can be used which is able to supply the microorganisms with sufficient amounts of carbon, so that acetate and/or ethanol, may be formed from the source of CO and/or CO₂.

Generally for the cell of the present invention the carbon source comprises at least 50% by weight, at least 70% by weight, particularly at least 90% by weight of CO₂ and/or CO, wherein the percentages by weight-% relate to all carbon sources that are available to the cell according to any aspect of the present invention. The carbon material source may be provided.

Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the mixed culture of the present invention.

According to any aspect of the present invention, the carbon source, also for the production of acetate and/or ethanol used in step (0) (see below) according to any aspect of the present invention may be synthesis gas. Synthesis gas can for example be produced as a by-product of coal gasification. Accordingly, the microorganism according to any aspect of the present invention may be capable of converting a substance which is a waste product into a valuable resource.

In another example, synthesis gas may be a by-product of gasification of widely available, low-cost agricultural raw materials for use with the mixed culture of the present invention to produce substituted and unsubstituted organic compounds.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Syngas may also be a product of electrolysis of CO₂. A skilled person would understand the suitable conditions to carry out electrolysis of CO₂ to produce syngas comprising CO in a desired amount.

Usually, a portion of the synthesis gas obtained from the gasification process is first processed in order to optimize product yields, and to avoid formation of tar. Cracking of the undesired tar and CO in the synthesis gas may be carried out using lime and/or dolomite. These processes are described in detail in for example, Reed, 1981.

The overall efficiency, alkanoic acid and/or ester thereof productivity and/or overall carbon capture of the method of the present invention may be dependent on the stoichiometry of the CO₂, CO, and H₂ in the continuous gas flow. The continuous gas flows applied may be of composition CO₂ and H₂. In particular, in the continuous gas flow, concentration range of CO₂ may be about 10-50%, in particular 3% by weight and Hz would be within 44% to 84%, in particular, 64 to 66.04% by weight. In another example, the continuous gas flow can also comprise inert gases like N₂, up to a N₂ concentration of 50% by weight.

Mixtures of sources can be used as a carbon source.

According to any aspect of the present invention, a reducing agent, for example hydrogen may be supplied together with the carbon source. In particular, this hydrogen may be supplied when the C and/or CO₂ is supplied and/or used. In one example, the hydrogen gas is part of the synthesis gas present according to any aspect of the present invention. In another example, where the hydrogen gas in the synthesis gas is insufficient for the method of the present invention, additional hydrogen gas may be supplied.

In one example, the alkanoic acid is hexanoic acid. More in particular, the carbon source comprising CO and/or CO₂ contacts the cells in a continuous gas flow. Even more in particular, the continuous gas flow comprises synthesis gas. These gases may be supplied for example using nozzles that open up into the aqueous medium, frits, membranes within the pipe supplying the gas into the aqueous medium and the like.

A skilled person would understand that it may be necessary to monitor the composition and flow rates of the streams at relevant intervals. Control of the composition of the stream can be achieved by varying the proportions of the constituent streams to achieve a target or desirable composition. The composition and flow rate of the blended stream can be monitored by any means known in the art. In one example, the system is adapted to continuously monitor the flow rates and compositions of at least two streams and combine them to produce a single blended substrate stream in a continuous gas flow of optimal composition, and means for passing the optimised substrate stream to the fermenter.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep and/or culture the cells, for example LB medium in the case of E. coli, ATCC1754-Medium may be used in the case of C. ljungdahlii. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium. The cells are incubated with the carbon source sufficiently long enough to produce the desired product. For example for at least 1, 2, 4, 5, 10 or 20 hours. The temperature chosen must be such that the cells according to any aspect of the present invention remains catalytically competent and/or metabolically active, for example 10 to 42° C., preferably 30 to 40° C., in particular, 32 to 38° C. in case the cell is a C. ljungdahlii cell. The aqueous medium according to any aspect of the present invention also includes the medium in which the alkanoic acid and/or ester thereof is produced. It mainly refers to a medium where the solution comprises substantially water. In one example, the aqueous medium in which the cells are used to produce the alkanoic acid and/or ester thereof is the very medium which contacts the extraction medium for extraction of the alkanoic acid and/or ester thereof.

In particular, the mixture of the microorganism and the carbon source according to any aspect of the present invention may be employed in any known bioreactor or fermenter to carry out any aspect of the present invention. In one example, the complete method according to any aspect of the present invention that begins with the production of the alkanoic acid and/or ester thereof and ends with the extraction of the alkanoic acid and/or ester thereof takes place in a single container. There may therefore be no separation step between the step of producing alkanoic acid and/or ester thereof and the step of extracting the alkanoic acid and/or ester thereof. This saves time and costs. In particular, during the fermentation process, the microorganism may be grown in the aqueous medium and in the presence of the extraction medium. The method according to any aspect of the present invention thus provides for a one pot means of producing alkanoic acids and/or ester thereof. Also, since the alkanoic acid and/or ester thereof is being extracted as it is produced, no end-product inhibition takes place, ensuring that the yield of alkanoic acid and/or ester thereof is maintained. A further step of separation may be carried out to remove the alkanoic acid and/or ester thereof. Any separation method known in the art such as using a funnel, column, distillation and the like may be used. The remaining extracting medium and/or the cells may then be recycled.

In another example, the extraction process may take place as a separate step and/or in another pot. After fermentation has taken place, where the desired alkanoic acid and/or ester thereof to be extracted has already been produced, the extracting medium according to any aspect of the present invention may be added to the fermentation medium or the fermentation medium may be added to a pot comprising the extracting medium. The desired alkanoic acid and/or ester thereof may then be extracted by any separation method known in the art such as using a funnel, column, distillation and the like. The remaining extracting medium may then be recycled.

Another advantage of the method is that the extracting medium may be recycled. Therefore, once the alkanoic acid and/or ester thereof is separated from extraction medium, the extraction medium can be recycled and reused, reducing waste.

According to another aspect of the present invention, there is provided a use of a mixture of at least one alkyl-phosphine oxide, preferably Trioctylphosphine oxide (TOPO), and alkane for extracting an alkanoic acid from an aqueous medium wherein the alkane comprises at least 12 carbon atoms. In particular, the alkane may comprise 12 to 18 carbon atoms. More in particular, the alkane may be hexadecane. Even more in particular, the alkanoic acid and/or ester thereof is selected from the group consisting of alkanoic acids with 4 to 16 carbon atoms. In one example, the alkanoic acid may be a hexanoic acid.

In a preferred method according to the instant invention ethanol and/or acetate is used as a starting material. This preferred method according to the instant invention extracts the alkanoic acid and/or ester thereof produced from ethanol and/or acetate comprises step (0) before step (a):
(0) contacting the ethanol and/or acetate with at least one microorganism capable of carrying out carbon chain elongation in the aqueous medium to produce the alkanoic acid and/or an ester thereof from the ethanol and/or acetate.

According to a preferred method according to the instant invention the aqueous medium after step (b) of separating the alkanoic acid and/or an ester thereof, may be recycled back into step (0). This step of recycling allows for the microorganisms to be recycled and reused as the extracting medium according to the present invention is not toxic to the microorganisms. This step of recycling the aqueous medium in the method according to the present invention has the further advantage of enabling the residue of the alkanoic acid and/or an ester thereof, which was not at first instance extracted from steps (a) and (b) in the first cycle, to be given a chance to be extracted a further time or as many times as the aqueous medium is recycled.

The microorganism in (0) capable of carrying out carbon chain elongation to produce the alkanoic acid may be any organism that may be capable of carbon-chain elongation (compare Jeon et al. Biotechnol Biofuels (2016) 9:129). The carbon chain elongation pathway is also disclosed in Seedorf, H., et al., 2008. The microorganisms according to any aspect of the present invention may also include microorganisms which in their wild-type form are not capable of carbon chain elongation, but have acquired this trait as a result of genetic modification. In particular, the microorganism in (0) may be selected from the group consisting of Clostridium carboxidivorans, Clostridium kluyveri and C. pharus. In particular, the microorganism according to any aspect of the present invention may be Clostridium kluyveri.

In step (0) according to any aspect of the present invention, ethanol and/or acetate is contacted with at least one microorganism capable of carrying out carbon chain elongation to produce the alkanoic acid and/or an ester thereof from the ethanol and/or acetate. In one example, the carbon source may be ethanol in combination with at least one other carbon source selected from the group consisting of acetate, propionate, butyrate, isobutyrate, valerate and hexanoate. In particular, the carbon source may be ethanol and acetate. In another example, the carbon source may be a combination of propionic acid and ethanol, acetate and ethanol, isobutyric acid and ethanol or butyric acid and ethanol. In one example, the carbon substrate may be ethanol alone. In another example, the carbon substrate may be acetate alone.

The source of acetate and/or ethanol may vary depending on availability. In one example, the ethanol and/or acetate may be the product of fermentation of synthesis gas or any carbohydrate known in the art. In particular, the carbon source for acetate and/or ethanol production may be selected from the group consisting of alcohols, aldehydes, glucose, sucrose, fructose, dextrose, lactose, xylose, pentose, polyol, hexose, ethanol and synthesis gas. Mixtures of sources can be used as a carbon source.

Even more in particular, the carbon source may be synthesis gas. The synthesis gas may be converted to ethanol and/or acetate in the presence of at least one acetogenic bacteria.

In one example, the production of the alkanoic acid and/or ester thereof is from acetate and/or ethanol which is from synthesis gas and may involve the use of the acetogenic bacteria in conjunction with a microorganism capable of carbon chain elongation. For example, Clostridium ljungdahlii may be used simultaneously with Clostridium kluyveri. In another example, a single acetogenic cell may be capable of the activity of both organisms.

For example, the acetogenic bacteria may be C. carboxidivorans which may be capable of carrying out both the Wood-Ljungdahl pathway and the carbon chain elongation pathway.

The ethanol and/or acetate used in step (0) according to any aspect of the present invention may be a product of fermentation of synthesis gas or may be obtained through other means.

The ethanol and/or acetate may then be brought into contact with the microorganism in step (0).

The term “contacting”, as used herein, means bringing about direct contact between the microorganism and the ethanol and/or acetate. In one example, ethanol is the carbon source and the contacting in step (0) involves contacting the ethanol with the microorganism of step (0). The contact may be a direct contact or an indirect one that may include a membrane or the like separating the cells from the ethanol or where the cells and the ethanol may be kept in two different compartments etc. For example, in step (a) the alkanoic acid and/or ester thereof, and the extracting medium, may be in different compartments.

According to any aspect of the present invention, where the extraction is carried out in step (a) as fermentation takes place in step (0), the time for extraction may be equivalent to the time of fermentation.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Clostridium kluyveri Forming Butyric Acid from Acetate and Ethanol

For the biotransformation of ethanol and acetate to butyric acid the bacterium Clostridium kluyveri was used. All cultivation steps were carried out under anaerobic conditions in pressure-resistant glass bottles that can be closed airtight with a butyl rubber stopper.

For the preculture 100 ml of DMSZ52 medium (pH=7.0; 10 g/L K-acetate, 0.31 g/L K₂HPO₄, 0.23 g/L KH₂PO₄, 0.25 g/l NH₄Cl, 0.20 g/l MgSO₄x7 H₂O, 1 g/L yeast extract, 0.50 mg/L resazurin, 10 μl/l HCl (25%, 7.7 M), 1.5 mg/L FeCl₂x4H₂O, 70 μg/L ZnCl₂x7H₂O, 100 μg/L MnCl₂x4H₂O, 6 μg/L H₃BO₃, 190 μg/L CoCl₂x6H₂O, 2 μg/L CuCl₂x6H₂O, 24 μg/L NiCl₂x6H₂O, 36 μg/L Na2MO₄x2H₂O, 0.5 mg/L NaOH, 3 μg/L Na₂SeO₃x5H₂O, 4 μg/L Na₂WO₄x2H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine —HClx2H₂O, 20 ml/L ethanol, 2.5 g/L NaHCO₃, 0.25 g/L cysteine-HClxH₂O, 0.25 g/L Na₂Sx9H₂O) in a 250 ml bottle were inoculated with 5 ml of a frozen cryoculture of Clostridium kluyveri and incubated at 37° C. for 144 h to an OD_{600 nm}>0.2.

For the main culture 200 ml of fresh DMSZ52 medium in a 500 ml bottle were inoculated with centrifuged cells from the preculture to an OD_{600 nm} of 0.1. This growing culture was incubated at 37° C. for 27 h to an OD_{600 nm}>0.6. Then the cell suspension was centrifuged, washed with production buffer (pH 6.0; 8.32 g/L K-acetate, 0.5 g/l ethanol) and centrifuged again.

For the production culture, 200 ml of production buffer in a 500 ml bottle was inoculated with the washed cells from the main culture to an OD_{600 nm} of 0.2. The culture was capped with a butyl rubber stopper and incubated for 71 h at 37° C. and 100 rpm in an open water shaking bath. At the start and end of the culturing period, samples were taken. These were tested for optical density, pH and the different analytes (tested by NMR).

The results showed that in the production phase the amount of acetate decreased from 5.5 g/l to 5.0 g/l and the amount of ethanol decreased from 0.5 g/l to 0.0 g/l. Also, the concentration of butyric acid was increased from 0.05 g/l to 0.8 g/l and the concentration of hexanoic acid was increased from 0.005 g/l to 0.1 g/l.

### Example 2

### Clostridium kluyveri Forming Hexanoic Acid from Acetate and Ethanol

For the biotransformation of ethanol and acetate to hexanoic acid the bacterium Clostridium kluyveri was used. All cultivation steps were carried out under anaerobic conditions in pressure-resistant glass bottles that can be closed airtight with a butyl rubber stopper.

For the preculture 100 ml of DMSZ52 medium (pH=7.0; 10 g/L K-acetate, 0.31 g/L K₂HPO₄, 0.23 g/L KH₂PO₄, 0.25 g/l NH₄Cl, 0.20 g/l MgSO₄x7H₂O, 1 g/L yeast extract, 0.50 mg/L resazurin, 10 μl/l HCl (25%, 7.7 M), 1.5 mg/L FeCl₂x4H₂O, 70 μg/L ZnCl₂x7H₂O, 100 μg/L MnCl₂x4H₂O, 6 μg/L H₃BO₃, 190 μg/L CoCl₂x6H₂O, 2 μg/L CuCl₂x6H₂O, 24 μg/L NiCl₂x6H₂O, 36 μg/L Na₂MO₄x2H₂O, 0.5 mg/L NaOH, 3 μg/L Na₂SeO₃x5H2O, 4 μg/L Na₂WO₄x2H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine -HClx2H₂O, 20 ml/L ethanol, 2.5 g/L NaHCO₃, 0.25 g/L cysteine-HClxH₂O, 0.25 g/L Na₂Sx9H₂O) in a 250 ml bottle were inoculated with 5 ml of a frozen cryoculture of Clostridium kluyveri and incubated at 37° C. for 144 h to an OD_{600 nm}>0.2.

For the main culture 200 ml of fresh DMSZ52 medium in a 500 ml bottle were inoculated with centrifuged cells from the preculture to an OD_{600 nm} of 0.1. This growing culture was incubated at 37° C. for 27 h to an OD_{600 nm}>0.6. Then the cell suspension was centrifuged, washed with production buffer (pH 6.0; 0.832 g/L K-acetate, 5.0 g/l ethanol) and centrifuged again.

For the production culture, 200 ml of production buffer in a 500 ml bottle was inoculated with the washed cells from the main culture to an OD_{600 nm} of 0.2. The culture was capped with a butyl rubber stopper and incubated for 71 h at 37° C. and 100 rpm in an open water shaking bath. At the start and end of the culturing period, samples were taken. These were tested for optical density, pH and the different analytes (tested by NMR).

The results showed that in the production phase the amount of acetate decreased from 0.54 g/l to 0.03 g/l and the amount of ethanol decreased from 5.6 g/l to 4.9 g/l. Also, the concentration of butyric acid was increased from 0.05 g/l to 0.28 g/l and the concentration of hexanoic acid was increased from 0.03 g/l to 0.79 g/l.

### Example 3

### Clostridium kluyveri Forming Hexanoic Acid from Butyric Acid and Ethanol

For the biotransformation of ethanol and butyric acid to hexanoic acid the bacterium Clostridium kluyveri was used. All cultivation steps were carried out under anaerobic conditions in pressure-resistant glass bottles that can be closed airtight with a butyl rubber stopper.

For the preculture 100 ml of DMSZ52 medium (pH=7.0; 10 g/L K-acetate, 0.31 g/L K₂HPO₄, 0.23 g/L KH₂PO₄, 0.25 g/l NH₄Cl, 0.20 g/l MgSO₄x7 H₂O, 1 g/L yeast extract, 0.50 mg/L resazurin, 10 μl/l HCl (25%, 7.7 M), 1.5 mg/L FeCl₂x4H₂O, 70 μg/L ZnCl₂x7H₂O, 100 μg/L MnCl₂x4H₂O, 6 μg/L H₃BO, 190 μg/L CoCl₂x6H₂O, 2 μg/L CuCl₂x6H₂O, 24 μg/L NiCl₂x6H₂O, 36 μg/L Na₂MO₄x2H₂O, 0.5 mg/L NaOH, 3 μg/L Na₂SeO₃x5H₂O, 4 μg/L Na₂WO₄x2H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCIx2H₂O, 20 ml/L ethanol, 2.5 g/L NaHCO₃, 0.25 g/L cysteine-HCIxH₂O, 0.25 g/L Na₂Sx9H₂O) in a 250 ml bottle were inoculated with 5 ml of a frozen cryoculture of Clostridium kluyveri and incubated at 37° C. for 144 h to an OD_{600 nm}>0.3.

For the main culture 200 ml of fresh DMSZ52 medium in a 500 ml bottle were inoculated with centrifuged cells from the preculture to an OD_{600 nm} of 0.1. This growing culture was incubated at 37° C. for 25 h to an OD_{600 nm}>0.4. Then the cell suspension was centrifuged, washed with production buffer (pH 6.16; 4.16 g/L K-acetate, 10.0 g/l ethanol) and centrifuged again.

For the production cultures, 200 ml of production buffer in a 500 ml bottle was inoculated with the washed cells from the main culture to an OD_{600 nm} of 0.2. In a first culture, at the beginning 1.0 g/l butyric acid was added to the production buffer, in a second culture, no butyric acid was added to the production buffer. The cultures were capped with a butyl rubber stopper and incubated for 71 h at 37° C. and 100 rpm in an open water shaking bath. At the start and end of the culturing period, samples were taken. These were tested for optical density, pH and the different analytes (tested by NMR).

The results showed that in the production phase of the butyric acid supplemented culture the amount of acetate decreased from 3.1 g/l to 1.1 g/l and the amount of ethanol decreased from 10.6 g/l to 7.5 g/l. Also, the concentration of butyric acid was increased from 1.2 g/l to 2.2 g/l and the concentration of hexanoic acid was increased from 0.04 g/l to 2.30 g/l.

In the production phase of the non-supplemented culture the amount of acetate decreased from 3.0 g/l to 1.3 g/l and the amount of ethanol decreased from 10.2 g/l to 8.2 g/l. Also, the concentration of butyric acid was increased from 0.1 g/l to 1.7 g/l and the concentration of hexanoic acid was increased from 0.01 g/l to 1.40 g/l.

### Example 4

### Cultivation of Clostridium kluyveri in Presence of Decane and TOPO

The bacterium Clostridium kluyveri DSM555 (German DSMZ) was cultivated for the biotransformation of ethanol and acetate to hexanoic acid. For the inSitu extraction of the produced hexanoic acid a mixture of decane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄ X 7 H₂O, 10 μl/L HCl (7.7 M), 1.5 mg/L FeCl₂ X 4 H₂O, 36 μg/L ZnCl₂, 64 μg/L MnCl₂ X 4 H₂O, 6 μg/L H₃BO₃, 190 μg/L CoCl₂ X 6 H₂O, 1.2 μg/L CuCl₂ X 6 H₂O, 24 μg/L NiCl₂ X 6 H₂O, 36 μg/L Na₂MO₄ X 2 H₂O, 0.5 mg/L NaOH, 3 μg/L Na₂SeO₃ X 5 H₂O, 4 μg/L Na₂WO₄ X 2 H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCl x 2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD_{600 nm} of 0.1. The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously fed to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle was inoculated with centrifuged cells from the preculture to an OD_{600 nm} of 0.1. Additional 1 ml of a mixture of 6% (w/w) TOPO in decane was added. The culture was capped with a butyl rubber stopper and incubated at 37° C. and 150 rpm in an open water bath shaker for 43 h under 100% CO₂ atmosphere. During cultivation several 5 mL samples were taken to determinate OD_{600 nm}, pH and product formation. The determination of the product concentrations was performed by semi-quantitative 1H-NMR spectroscopy. As an internal quantification standard sodium trimethylsilylpropionate (T(M)SP) was used.

During the main cultivation the concentration of butyrate increased from 0.14 g/L to 2.12 g/L and the concentration of hexanoate increased from 0.22 g/L to 0.91 g/L, whereas the concentration of ethanol decreased from 15.04 to 11.98 g/l and the concentration of acetate decreased from 6.01 to 4.23 g/L.

The OD_{600 nm} decreased during this time from 0.111 to 0.076.

### Example 5

### Cultivation of Clostridium kluyveri in Presence of Tetradecane and TOPO

The bacterium Clostridium kluyveri was cultivated for the biotransformation of ethanol and acetate to hexanoic acid. For the inSitu extraction of the produced hexanoic acid a mixture of tetradecane with trioctylphosphineoxide (TOPO) was added to the cultivation. All cultivation steps were carried out under anaerobic conditions in pressure-resistant glass bottles that can be closed airtight with a butyl rubber stopper.

The precultivation of Clostridium kluyveri was carried out in a 1000 mL pressure-resistant glass bottle in 250 ml of EvoDM24 medium (pH 5.5; 0.429 g/L Mg-acetate, 0.164 g/l Na-acetate, 0.016 g/L Ca-acetate, 2.454 g/l K-acetate, 0.107 mL/L H₃PO₄ (8.5%), 0.7 g/L NH₄acetate, 0.35 mg/L Co-acetate, 1.245 mg/L Ni-acetate, 20 μg/L d-biotin, 20 μg/L folic acid,10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 0.702 mg/L (NH₄)2Fe(SO₄)₂x4H₂O, 1 ml/L KS-acetate (93.5 mM), 20 mL/L ethanol, 0.37 g/L acetic acid) at 37° C., 150 rpm and a ventilation rate of 1 L/h with a mixture of 25% CO₂ and 75% N₂ in an open water bath shake. The gas was discharged into the headspace of the reactor. The pH was hold at 5.5 by automatic addition of 2.5 M NH₃ solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor and hold an OD_{600 nm} of ˜1.5.

For the main culture 100 ml of Veri01 medium (pH 6.5; 10 g/L potassium acetate, 0.31 g/L K₂HPO₄, 0.23 g/L KH₂PO₄, 0.25 g/L NH₄Cl, 0.20 g/L MgSO₄X7H₂O, 10 μl/L HCl (7.7 M), 1.5 mg/L FeCl₂X4H₂O, 36 μg/L ZnCl₂, 64 μg/L MnCl₂X4H₂O, 6 μg/L H₃BO₃, 190 μg/L CoCl₂X6H₂O, 1.2 μg/L CuCl₂X6H₂O, 24 μg/L NiCl₂X6H₂O, 36 μg/L Na₂MO₄X2H₂O, 0.5 mg/L NaOH, 3 μg/L Na₂SeO₃X5H₂O, 4 μg/L Na₂WO₄X2H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HClx2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine, 2.5 mL/L HCL 25%) in a 250 ml bottle were inoculated with centrifuged cells from the preculture to an OD_{600 nm} of 0.1. Additional 1 ml of a mixture of 6% (w/w) TOPO in tetradecane was added. The culture was capped with a butyl rubber stopper and incubated at 37° C. and 150 rpm in an open water bath shaker for 47 h under 100% CO₂ atmosphere.

During cultivation several 5 mL samples were taken to determinate OD_{600 nm}, pH and product formation. The determination of the product concentrations was performed by semiquantitative 1H-NMR spectroscopy. As an internal quantification standard sodium trimethylsilylpropionate (T(M)SP) was used.

During the main cultivation the concentration of butyrate increased from 0.05 g/L to 3.78 g/L and the concentration of hexanoate increased from 0.09 g/L to 4.93 g/L, whereas the concentration of ethanol decreased from 15.52 to 9.36 g/l and the concentration of acetate decreased from 6.36 to 2.49 g/L.

The OD_{600 nm} increased during this time from 0.095 to 0.685.

### Example 6

### Cultivation of Clostridium kluyveri in Presence of Hexadecane and TOPO

The bacterium Clostridium kluyveri was cultivated for the biotransformation of ethanol and acetate to hexanoic acid. For the inSitu extraction of the produced hexanoic acid a mixture of hexadecane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄ X 7 H₂O, 10 μl/L HCl (7.7 M), 1.5 mg/L FeCl₂ X 4 H₂O, 36 μg/L ZnCl₂, 64 μg/L MnCl₂ X 4 H₂O, 6 μg/L H₃BO₃, 190 μg/L CoCl₂ X 6 H₂O, 1.2 μg/L CuCl₂ X 6 H₂O, 24 μg/L NiCl₂ X 6 H₂O, 36 μg/L Na₂MO₄ X 2 H₂O, 0.5 mg/L NaOH, 3 μg/L Na₂SeO₃ X 5 H₂O, 4 μg/L Na₂WO₄ X 2 H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCl x 2 H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD_{600 nm} of 0.1.

The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously fed to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle was inoculated with centrifuged cells from the preculture to an OD_{600 nm} of 0.1. Additional 1 ml of a mixture of 6% (w/w) TOPO in hexadecane was added. The culture was capped with a butyl rubber stopper and incubated at 37° C. and 150 rpm in an open water bath shaker for 43 h under 100% CO₂ atmosphere.

During cultivation several 5 mL samples were taken to determinate OD_{600 nm}, pH and product formation. The determination of the product concentrations was performed by semi-quantitative 1H-NMR spectroscopy. As an internal quantification standard sodium trimethylsilylpropionate (T(M)SP) was used.

During the main cultivation the concentration of butyrate increased from 0.14 g/L to 2.86 g/L and the concentration of hexanoate increased from 0.20 g/L to 2.37 g/L, whereas the concentration of ethanol decreased from 14.59 to 10.24 g/l and the concentration of acetate decreased from 5.87 to 3.32 g/L.

The OD_{600 nm} increased during this time from 0.091 to 0.256.

### Example 7

### Cultivation of Clostridium kluyveri in Presence of Heptadecane and TOPO

The bacterium Clostridium kluyveri was cultivated for the biotransformation of ethanol and acetate to hexanoic acid. For the inSitu extraction of the produced hexanoic acid a mixture of heptadecane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄ X 7 H2O, 10 μl/L HCl (7.7 M), 1.5 mg/L FeCl₂ X 4 H₂O, 36 μg/L ZnCl₂, 64 μg/L MnCl₂ X 4 H₂O, 6 μg/L H₃BO₃, 190 μg/L CoCl₂ X 6 H₂O, 1.2 μg/L CuCl₂ X 6 H₂O, 24 μg/L NiCl₂ X 6 H₂O, 36 μg/L Na₂MO₄ X 2 H₂O, 0.5 mg/L NaOH, 3 μg/L Na₂SeO₃ X 5 H₂O, 4 μg/L Na₂WO₄X2H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCl x 2 H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD_{600 nm} of 0.1.

The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle were inoculated with centrifuged cells from the preculture to an OD_{600 nm} of 0.1. Additional 1 ml of a mixture of 6% (w/w) TOPO in heptadecane was added. The culture was capped with a butyl rubber stopper and incubated at 37° C. and 150 rpm in an open water bath shaker for 43 h under 100% CO₂ atmosphere.

During cultivation several 5 mL samples were taken to determinate OD_{600 nm}, pH and product formation. The determination of the product concentrations was performed by semiquantitative 1H-NMR spectroscopy. As an internal quantification standard sodium trimethylsilylpropionate (T(M)SP) was used.

During the main cultivation the concentration of butyrate increased from 0.15 g/L to 2.82 g/L and the concentration of hexanoate increased from 0.19 g/L to 2.85 g/L, whereas the concentration of ethanol decreased from 14.34 to 9.58 g/l and the concentration of acetate decreased from 5.88 to 3.20 g/L.

The OD_{600 nm} increased during this time from 0.083 to 0.363.

### Example 8

### Cultivation of Clostridium kluyveri in Presence of Dodecane and TOPO

The bacterium Clostridium kluyveri was cultivated for the biotransformation of ethanol and acetate to hexanoic acid. For the inSitu extraction of the produced hexanoic acid a mixture of dodecane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄ X 7 H₂O, 10 μl/L HCl (7.7 M), 1.5 mg/L FeCl₂ X 4 H₂O, 36 μg/L ZnCl₂, 64 μg/L MnCl₂ X 4 H₂O, 6 μg/L H3O₃, 190 μg/L CoCl₂ X 6 H₂O, 1.2 μg/L CuCl₂ X 6 H₂O, 24 μg/L NiCl2 X 6 H2O, 36 μg/L Na₂MO₄ X 2 H₂O, 0.5 mg/L NaOH, 3 μg/L Na₂SeO₃ X 5 H₂O, 4 μg/L Na₂WO₄ X 2 H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/I thiamine-HCl x 2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD_{600 nm} of 0.1.

The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle were inoculated with centrifuged cells from the preculture to an OD_{600 nm} of 0.1. Additional 1 ml of a mixture of 6% (w/w) TOPO in dodecane was added. The culture was capped with a butyl rubber stopper and incubated at 37° C. and 150 rpm in an open water bath shaker for 43 h under 100% CO₂ atmosphere.

During cultivation several 5 mL samples were taken to determinate OD_{600 nm}, pH and product formation. The determination of the product concentrations was performed by semiquantitative 1H-NMR spectroscopy. As an internal quantification standard sodium trimethylsilylpropionate (T(M)SP) was used.

During the main cultivation the concentration of butyrate increased from 0.14 g/L to 2.62 g/L and the concentration of hexanoate increased from 0.22 g/L to 2.05 g/L, whereas the concentration of ethanol decreased from 14.62 to 10.64 g/I and the concentration of acetate decreased from 5.92 to 3.54 g/L.

The OD_{600 nm} increased during this time from 0.091 to 0.259.

### Example 9

### Determination of the Distribution Coefficient for Hexanoic Acid Between Water and a Mixture of Hexadecane and TOPO

During all stages of the experiment, samples from both phases were taken for determination of pH and concentration of hexanoic acid by high performance liquid chromatography (HPLC). 100 g of an aqueous solution of 5 g/kg hexanoic acid and 33 g of a mixture of 6% trioctylphosphinoxide (TOPO) in hexadecane were filled in a separatory funnel and mixed for 1 minute at 37° C. Then the funnel was placed in a tripod ring and the emulsion was left to stand to separate spontaneously. The pH of the aqueous phase was measured. Then 1M NaOH solution was added to the funnel and mixed. The step of separation and sampling was repeated until a pH of 6.2 in the aqueous phase was reached. Samples from both phases were taken for later analysis at this point. The aqueous phase could be analyzed directly by HPLC. For the analysis of the organic phase the diluted hexanoic acid was first re-extracted to water (pH 12.0 by addition of 1 M NaOH) and then analyzed by HPLC. The distribution coefficient K_{D} of hexanoic acid in the system of water and 6% TOPO in hexadecane was calculated from the concentrations of hexanoic acid in both phases.

$$K ( D ) = \frac{c ( Hex , organic phase )}{c ( Hex , aqueous phase )}$$

The K_{D} for hexanoic acid in the system of water and 6% TOPO in hexadecane at pH 6.2 was 4.7.

### Example 10

### Determination of the Distribution Coefficient for Hexanoic Acid Between Water and a Mixture of Heptadecane and TOPO

During all stages of the experiment, samples from both phases were taken for determination of pH and concentration of hexanoic acid by high performance liquid chromatography (HPLC). 100 g of an aqueous solution of 5 g/kg hexanoic acid and 33 g of a mixture of 6% trioctylphosphinoxide (TOPO) in heptadecane were filled in a separatory funnel and mixed for 1 minute at 37° C. Then the funnel was placed in a tripod ring and the emulsion was left to stand to separate spontaneously. The pH of the aqueous phase was measured. 1M NaOH solution was added to the funnel and mixed. The step of separation and sampling was repeated until a pH of 6.2 in the aqueous phase was reached. Samples from both phases were taken for later analysis at this point. The aqueous phase could be analyzed directly by HPLC. For the analysis of the organic phase the diluted hexanoic acid was first re-extracted to water (pH 12.0 by addition of 1 M NaOH) and then analyzed by HPLC. The distribution coefficient K_{D} of hexanoic acid in the system of water and 6% TOPO in heptadecane was calculated from the concentrations of hexanoic acid in both phases.

$$K ( D ) = \frac{c ( Hex , organic phase )}{c ( Hex , aqueous phase )}$$

The K_{D} for hexanoic acid in the system water and 6% TOPO in heptadecane at pH 6.2 was 5.0.

### Example 11

### Determination of the Distribution Coefficient for Hexanoic Acid Between Water and a Mixture of Tetradecane and TOPO

During all stages of the experiment, samples from both phases were taken for determination of pH and concentration of hexanoic acid by high performance liquid chromatography (HPLC). 130 g of an aqueous solution of 5 g/kg hexanoic acid plus 0.5 g/kg acetic acid and 15 g of a mixture of 6% trioctylphosphinoxid (TOPO) in tetradecane were filled in a separatory funnel and mixed for 1 minute at 37° C. Then the funnel was placed in a tripod ring and the emulsion was led stand to separate spontaneously. The pH of the aqueous phase was measured. 1M NaOH solution was added to the funnel and mixed. The step of separation and sampling was repeated until a pH of 6.2 in the aqueous phase was reached. Samples from both phases were taken for later analysis at this point. The aqueous phase could be analyzed directly by HPLC. For the analysis of the organic phase the diluted hexanoic acid was first re-extracted to water (pH 12.0 by addition of 1 M NaOH) and then analyzed by HPLC. The distribution coefficient K_{D} of hexanoic acid in the system water and 6% TOPO in tetradecane was calculated from the concentrations of hexanoic acid in both phases.

$$K ( D ) = \frac{c ( Hex , organic phase )}{c ( Hex , aqueous phase )}$$

The K_{D} for hexanoic acid in the system water and 6% TOPO in tetradecane at pH 6.9 was 1.3.

### Example 12

### Cultivation of Clostridium kluyveri with inSitu Extraction of Hexanoic Acid

The bacterium Clostridium kluyveri was cultivated for the biotransformation of ethanol and acetate to hexanoic acid. For the inSitu extraction of the produced hexanoic acid a mixture of tetradecane with trioctylphosphineoxide (TOPO) was continuously passed through the cultivation. All cultivation steps were carried out under anaerobic conditions in pressure-resistant glass bottles that can be closed airtight with a butyl rubber stopper.

The precultivation of Clostridium kluyveri was carried out in a 1000 mL pressure-resistant glass bottle in 250 ml of EvoDM45 medium (pH 5.5; 0.004 g/L Mg-acetate, 0.164 g/l Na-acetate, 0.016 g/L Ca-acetate, 0.25 g/l K-acetate, 0.107 mL/L H₃PO₄ (8.5%), 2.92 g/L NH₄acetate, 0.35 mg/L Co-acetate, 1.245 mg/L Ni-acetate, 20 μg/L d-biotin, 20 μg/L folic acid, 10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μ/L lipoic acid, 0.702 mg/L (NH₄)₂Fe(SO₄)2 x 4 H₂O, 1 ml/L KS-acetate (93.5 mM), 20 mL/L ethanol, 0.37 g/L acetic acid) at 37° C., 150 rpm and a ventilation rate of 1 L/h with a mixture of 25% CO₂ and 75% N₂ in an open water bath shaker. The gas was discharged into the headspace of the reactor. The pH was hold at 5.5 by automatic addition of 2.5 M NH₃ solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor and hold an OD_{600 nm} of ˜1.5.

For the main culture 150 ml of EvoDM39 medium (pH 5.8; 0.429 g/L Mg-acetate, 0.164 g/I Na-acetate, 0.016 g/L Ca-acetate, 2.454 g/l K-acetate, 0.107 mL/L H₃PO₄ (8.5%), 1.01 mL/L acetic acid, 0.35 mg/L Co-acetate, 1.245 mg/L Ni-acetate, 20 μg/L d-biotin, 20 μg/L folic acid,10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 0.702 mg/L (NH₄)₂Fe(SO₄)₂×4 H₂O, 1 ml/L KS-acetate (93.5 mM), 20 mL/L ethanol, 8.8 mL NH₃ solution (2.5 mol/L), 27.75 ml/L acetic acid (144 g/L)) in a 1000 ml bottle were inoculated with 100 ml cell broth from the preculture to an OD_{600 nm} of 0.71.

The cultivation was carried out at 37° C., 150 rpm and a ventilation rate of 1 L/h with a mixture of 25% CO₂ and 75% N₂ in an open water bath shaker for 65 h. The gas was discharged into the headspace of the reactor. The pH was hold at 5.8 by automatic addition of 2.5 M NH₃ solution. Fresh medium was continuously feeded to the reactor with a dilution rate of 0.5 d⁻¹ and fermentation broth continuously removed from the reactor by holding an OD_{600 nm} of ˜0.5. Additional 120 g of a mixture of 6% (w/w) TOPO in tetradecane was added to the fermentation broth. Then this organic mixture was continuously feeded to the reactor and the organic phase also continuously removed from the reactor with a dilution rate of 1 d⁻¹.

During cultivation several 5 mL samples from both, the aqueous and the organic phase, were taken to determinate OD_{600 nm}, pH and product formation. The determination of the product concentrations was performed by semiquantitative 1H-NMR spectroscopy. As an internal quantification standard sodium trimethylsilylpropionate (T(M)SP) was used.

During the main cultivation in the aqueous phase a steady state concentration of 8.18 g/L ethanol, 3.20 g/L acetate, 1.81 g/L butyrate and 0.81 g/L hexanoate was reached. The OD_{600 nm} remained stable at 0.5. In the organic phase a steady state concentration of 0.43 g/kg ethanol, 0.08 g/kg acetate, 1.13 g/kg butyrate and 8.09 g/kg hexanoate was reached. After the experiment the cells remained viable while transferred to further cultivations.

The distribution coefficient K_{D} of the substrates and products in the system aqueous medium and 6% TOPO in tetradecane was calculated from the concentrations in both phases.

$$K ( D ) = \frac{c ( organic phase )}{c ( aqueous phase )}$$

The K_{D} in the steady state was 0.05 for ethanol, 0.03 for acetic acid, 0.62 for butyric acid and 9.99 for hexanoic acid.

1. A method of extracting an alkanoic acid and/or ester thereof from an aqueous medium, the method comprising:
(a) contacting the alkanoic acid and/or ester thereof in the aqueous medium with an extracting medium for a time sufficient to extract the alkanoic acid and/or ester thereof from the aqueous medium into the extracting medium, and
(b) separating the extracting medium with the extracted alkanoic acid and/or ester thereof from the aqueous medium,
wherein the extracting medium comprises:
a mixture of an alkyl-phosphine oxide, and an alkane,
wherein the alkane comprises at least 12 carbon atoms.

2. The method of claim 1, wherein the alkane comprises 12 to 18 carbon atoms.

3. The method of claim 1, wherein the alkane is hexadecane.

4. The method of claim 1, wherein the alkanoic acid and/or ester thereof is selected from the group consisting of alkanoic acids with 4 to 16 carbon atoms.

5. The method of claim 1, wherein the alkanoic acid is a hexanoic acid.

6. The method of claim 5, wherein the hexanoic acid is produced from synthesis gas by a method comprising:
contacting the synthesis gas with bacteria capable of carrying out the Wood-Ljungdahl pathway and ethanol-carboxylate fermentation to produce hexanoic acid.

7. The method of claim 6, wherein the bacteria is selected from the group consisting of Clostridium kluyveri and C. Carboxidivorans.

8. The method of claim 1, wherein a weight ratio of the alkyl-phosphine oxide to the alkane is between 1:100 to 1:10.

9. The method of claim 1, wherein a pH of the aqueous medium is maintained between 5.5 and 7.

10. The method of claim 1, wherein the extracting medium is recycled.

11-15. (canceled)
